(19) United States
(12) Patent Application Publication
(10) Pub. No.: US 2009/0098224 A1
(43) Pub. Date: Apr. 16, 2009

(54) Metabolic enhancing properties of theaflavins and thearubigins

(76) Inventors: Derek W. Cornelius, Cape Girardeau, MO (US); Gary L. Haynes, Scott City, MO (US)

Correspondence Address: Derek W. Cornelius, 251 Garnet Lane, Cape Girardeau, MO, 63701, US

(21) Appl. No.: 11/974,032

(22) Filed: Oct. 11, 2007

(51) Int. Cl. A61K 36/82; A23L 1/00; A61P 3/02; A61P 3/04; A23L 1/29

(52) U.S. Cl. 424/729; 426/648; 426/655

(57) ABSTRACT

Naturally occurring or synthetically derived theaflavins and thearubigins are disclosed which are orally active and have novel and specific metabolic enhancing effects in humans. The inventors have discovered that theaflavins and thearubigins decrease appetite, reduce adipose tissue, increase metabolism and energy levels and protect and enhance lean body mass.

### BACKGROUND OF THE INVENTION

Catechins derived from green tea have been known to increase metabolism and enhance weight loss for many years. They are included in many commercially available weight loss formulas. Although they do not exert a powerful effect, they do cause modest weight loss and possibly even some modest appetite suppression.

The catechins from green tea include epicatechin gallate (ECG), epigallocatechin gallate (EGCG), epigallocatechin (EGC), epicatechin (EC), and catechin gallate (CG). The most potent and important of these catechins in terms of increasing weight loss is CG and EGCG. There are many theories as to how these catechins exert their anti-obesity effect; however, the authors' believe that they mainly cause weight loss through inhibition of fatty acid synthase (FAS).

Fatty acid synthase is the single eukaryotic enzyme responsible for converting dietary carbohydrates into fats. This enzyme is thus critical in the induction, growth and maintenance of body fat. If this enzyme was effectively inhibited, it would dramatically impair the body's ability to gain and store body fat. A great amount of research has been accomplished in recent years in regards to fatty acid synthase and its inhibition. It is now known that it is possible to reduce adipose mass in certain mammals by carefully inhibiting this enzyme. Unfortunately, no fatty acid synthase inhibitors have been shown to be orally effective in humans.

It is known that EGCG and CG are inhibitors of fatty acid synthase but there is no conclusive evidence as to whether these catechins are active in-vivo in humans as FAS inhibitors or whether they are potent enough or work in the right tissues to exert a noticeable effect. Based on publicly available data as well as the inventors' own research, the inventors concluded that green tea was mainly exerting its positive effects on weight loss through inhibiting the fatty acid synthase enzyme. The inventors believe; however, that EGCG and CG showed only modest results, at best, because they were either poorly bioavailable or were not highly active in the right tissues.

The inventors therefore, searched for compounds that would work similarly to the catechins in green tea but would be much more potent via the oral route of delivery.

After testing dozens of compounds, the inventors discovered that theaflavins and thearubigins from black tea extract, within specific dosage ranges, exert the same type of effects as the catechins from green tea except that they are much more potent via the oral route of administration. Although other physiological mechanisms could be at work, the authors concluded that the theaflavins and thearubigins were exerting their positive metabolic enhancements through fatty acid synthase inhibition.

Although prior art demonstrates theaflavins and thearubigins to have a plethora of pharmacological properties, this is the first time that these compounds, when dosed orally to humans, have been disclosed to have the metabolic enhancing properties according to this invention. Likewise, prior art demonstrates that black tea may be useful for weight loss and increasing energy levels but these effects have never been attributed to the theaflavins and thearubigins.

Theaflavins and thearubigins are the predominant polyphenols in black tea. They are closely related polyphenols to the catechins in green tea. Black tea is produced from green tea via fermentation. In the fermentation process, theaflavins and thearubigins are produced from the catechins in green tea. The monomeric catechins undergo polyphenol oxidase-dependent oxidative polymerization to produce theaflavins and thearubigins. Theaflavins include theaflavin, theaflavin gallate, and theaflavin digallate. Thearubigins include a wide range of complex compounds that are difficult to characterize and identify.

The inventors have discovered new uses for the polyphenols of this invention including decreasing appetite for food, reducing adipose tissue, increasing metabolism and energy levels and protecting and enhancing lean body mass. The inventors believe that all of the above metabolic properties are attributable to the ability of the theaflavins and thearubigins to be an orally active and effective fatty acid synthase inhibitor.

Upon experimenting with theaflavins and thearubigins, the inventors immediately discovered that they had a profound appetite reducing effect. The inventors compared the potency of these polyphenols to the catechins in green tea. It was observed that although green tea barely affects appetite regardless of the dosage, the polyphenols in black tea, at an effective dosage, have a profound effect. Within 30 minutes of ingestion, those taking an effective amount of theaflavins and/or thearubigins notice a complete lack of hunger pangs. This effect is consistently noticed even if the person has not eaten food in many hours. Unlike other anorectic agents, such as amphetamines, the polyphenols of this invention do not cause any feelings of nausea. This is a significant advantage over other appetite reducing agents since a person consuming these polyphenols will not feel sickly.

The inventors believe that the polyphenols of this invention decrease appetite through a specific inhibition of fatty acid synthase. It is theorized that theaflavins and thearubigins specifically inhibit fatty acid synthase in the hypothalamus and other appetite control centers in the body. The inhibition of fatty acid synthase in these areas would allow the buildup of certain physiological substrates which would then create a negative feedback in terms of appetite.

Another novel characteristic of the polyphenols of this invention is their ability to reduce the amount of adipose tissue in a human. This effect is usually noticeable within 2-4 weeks of consuming these compounds. The inventors believe that this characteristic is directly related to the compounds' ability to inhibit fatty acid synthase throughout the entire body. In effect, the compounds of this invention cripple one of the main ways that a person's physiology uses to maintain and add adipose tissue . . . the result being vastly reduced adipose stores throughout the entire body.

The inventors theorized that if fatty acid synthase could be effectively inhibited throughout the body, then it would lead to a person having a higher metabolism and greater energy levels. The reason for this phenomenon would be that there would be a greater amount of energy substrates available for fuel. Instead of the carbohydrates being converted into adipose storage tissue, it would be available for immediate use as fuel. The greater amount of fuel substrates would be used for an increased metabolism and greater energy levels.

Upon testing theaflavins and thearubigins, it became evident that this theory was correct. The polyphenols of this invention give a person a noticeably increased metabolism and energy levels. The increased metabolism can be measured through various metabolic testing equipment or simply by measuring body temperature. The increased energy levels are perceptively noticeable by a person using the compounds of this invention in terms of increased feelings of wakefulness, ability to do work, and increased endurance.

This invention finally discloses that theaflavins and thearubigins have novel actions on muscle and lean body mass. The inventors believe that fatty acid synthase inhibition diverts nutrients away from adipose tissue for other uses. As previously mentioned, these nutrients can be used for an increased metabolism and increased energy levels. Likewise, the inventors theorize that these nutrients can be used to protect, maintain and build lean body mass.

Upon experimentation, the inventors discovered that theaflavins and thearubigins indeed have a positive impact on lean body mass. This effect is usually noticeable within one month of consuming an effective amount of the polyphenols of this invention. If a calorie surplus is consumed, muscle tissue can actually be gained over a period of time. If a calorie deficit is consumed, then the compounds of this invention have more of a sparing or protective effect on muscle tissue. Besides muscle tissue, theaflavins and thearubigins can also have a positive effect on skin and joint tissues.

Both theaflavins and thearubigins work equally well in terms of providing results as per the art of this invention. However, the inventors have found that administering these two polyphenols simultaneously provides results that are in excess of either theaflavins or thearubigins administered alone. The inventors are not sure as to which physiological mechanisms are responsible for this particular phenomenon, although they believe it may be due to theaflavins and thearubigins inhibiting FAS differently in various tissues. Different black tea extracts were subjected to experimentation in order to identify those providing the best results in the present invention. It was concluded that the best extracts contained a minimum of 10% theaflavins and 20% thearubigins. Furthermore, it was discovered that any ratio between 1:10 and 5:1 (theaflavins:thearubigins) would work well. The most preferred extracts contain a minimum of 10% theaflavins and 20% thearubigins and have a ratio between 1:1 and 1:8. Black tea extracts which are standardized for both theaflavins and thearubigins as per this invention are novel discoveries as there is no mention of these in prior art.

The theaflavins and thearubigins according to this invention may be derived from either natural or synthetic means. Currently the most efficient and cost effective means of obtaining these polyphenols is through extraction of black tea leaves, although other natural sources may be used. It is also possible to produce these compounds using synthetic or semi-synthetic techniques. For instance, catechins may be obtained in pure form from green tea and then semi-synthetically converted into specific theaflavins or thearubigins.

Effective human dosaging for the polyphenols of this invention is between 10 mg and 10,000 mg per day. A more effective range is between 100 mg and 2000 mg per day. Although the components of the present invention can be dosed once per day for a desired effect, we have found that it is best to divide the dosage into two or three equal amounts given eight to twelve hours apart. This helps to ensure steady blood values and an overall more powerful, consistent effect. Effective dosaging may be in the form of immediate or controlled release tablets, capsules, sachets, effervescent powder or tablets, softgels, liquid or other oral delivery systems known in the art.

### EXAMPLE 1

A black tea extract standardized for 40% theaflavins was given to 5 individuals at a dosage of 2 mg/kg three times daily 30 minutes before meals. Subjective effects on appetite were measured during the meal and 2 hours after the meal for a period of one week. In addition, subjective effects on energy levels throughout the day were measured for the same one week period. Although one subject did not appear to experience any appetite-reducing effects, the other 4 subjects reported significant improvements. The mean subjective reduction in appetite during the meal was 26%; 2 hours after the meal was 45%. In regards to the theaflavins improving energy levels, all five subjects reported noticeable improvements. The mean improvement in subjective energy levels was 9%.

### EXAMPLE 2

A second experiment was performed to measure the effects of theaflavins on adipose tissue and lean body mass. A black tea extract standardized for 20% theaflavins and 30% thearubigans was given to 4 individuals at a dosage of 5 mg/kg three times daily 30 minutes before meals. A fifth subject was the control. All subjects were asked to maintain their current diet and exercise regimens. Weekly measurements were recorded for fat mass and lean body mass.

TABLE 1
Fat Mass (in pounds)

| Subject | Week 1 | Week 2 | Week 3 | Week 4 |
|---|---|---|---|---|
| 1 | 0 | −.5 | −.1 | −1.25 |
| 2 | −1 | −1.5 | −2 | −2.5 |
| 3 | −1 | −1 | −.1.25 | −2 |
| 4 | −.5 | −1.25 | −1.5 | −1.5 |
| Control | 0 | 0 | 0 | 0 |

TABLE 2
Lean Body Mass (in pounds)

| Subject | Week 1 | Week 2 | Week 3 | Week 4 |
|---|---|---|---|---|
| 1 | 0 | .25 | .25 | 0 |
| 2 | −.25 | 0 | 0 | 0 |
| 3 | 0 | 0 | −.25 | 0 |
| 4 | .25 | .25 | .5 | .75 |
| Control | 0 | −.25 | 0 | −.25 |

The average fat mass loss after 4 weeks for the four subjects was 1.8 pounds while the control lost no weight during this time period. It is also readily evident from looking at the chart for lean body mass that the theaflavins and thearubigans had a significant effect at preserving or even increasing lean body mass even while the subject was losing adipose tissue.

Those skilled in the art will appreciate that numerous changes and modifications may be made to the preferred embodiments of the invention and that such changes and modifications may be made without departing from the spirit of the invention. It is therefore intended that the appended claims cover all such equivalent variations as fall within the true spirit and scope of the invention.

We claim:

1. A method of inducing weight loss, increasing metabolism and energy or increasing lean body mass in a human in need thereof comprising the administration of an effective amount of theaflavins or thearubigins.

2. The method of claim 1 in which the weight loss is caused by a reduction in appetite for food.

3. The method of claim 1 in which the weight loss is caused by a reduction in adipose tissue.

4. The method of claim 1 in which the effective amount according is between 10 mg and 10,000 mg per day.

5. The method of claim 4 in which the effective amount is between 100 mg and 2000 mg per day.

6. The method of claim 1 in which the effective dose is administered as a divided daily dose given 8 to 12 hours apart.

7. The method of claim 1 in which the effective dose is administered as a controlled release dosage form.

8. The method of claim 1 wherein the administration of the theaflavins or thearubigins inhibits fatty acid synthase.

9. A food or dietary supplement comprising an effective amount of a standardized extract comprising at least 10% theaflavins and at least 20% thearubigins.

10. The food or dietary supplement of claim 9 wherein the ratio of theaflavins to thearubigans ranges from 1:10 to 5:1 theaflavins to thearubigans.

11. The food or dietary supplement of claim 10 wherein the ratio of theaflavins to thearubigans ranges from 1:1 to 1:8.

12. The food or food supplement of claim 9 in which the total amount of theaflavins or thearubigans is between 10 mg and 10,000 mg.

13. The food or food supplement of claim 9 in which the total amount of theaflavins or thearubigans is between 100 mg and 2,000 mg.

14. The method of claim 14 in which the effective amount is between 100 mg and 2000 mg per day.

15. A method of abating hunger pangs, comprising the administration of an effective amount of theaflavins or thearubigins.

16. The method of claim 15 wherein the theaflavins or thearubigins are administered prior to a meal.

17. The method of claim 15 wherein the theaflavins or thearubigins are administered as a black tea extract.

18. The method of claim 17 wherein the black tea extract is standardized for at least 10% theaflavins or 20% thearubigans.

19. The method of claim 15 wherein the administration of theaflavins or thearubigins inhibits fatty acid synthase.

20. The method of claim 15 in which the effective amount is between 10 mg and 10,000 mg per day.

21. The method of claim 15 in which the effective amount is between 100 mg and 2000 mg per day.
